(19) United States
(12) Patent Application Publication
(10) Pub. No.: US 2025/0206702 A1
(43) Pub. Date: Jun. 26, 2025

(54) SYNTHESIS OF DOPAMINE D3 RECEPTOR SELECTIVE ANTAGONISTS/PARTIAL AGONISTS

(71) Applicant: THE UNITED STATES OF AMERICA, AS REPRESENTED BY THE SECRETARY, DEPARTMENT OF HEALTH & HUMAN SERVICES, Bethesda, MD (US)

(72) Inventors: Bangwei DING, Rockville, MD (US); Asaf Ragim ALIMARDANOV, Rockville, MD (US); Junfeng HUANG, Rockville, MD (US)

(21) Appl. No.: 18/851,859

(22) PCT Filed: Mar. 27, 2023
(86) PCT No.: PCT/US2023/016357

§ 371 Date: Sep. 27, 2024

Related U.S. Application Data
(60) Provisional application No. 63/325,313, filed on Mar. 30, 2022.

(51) Int. Cl. C07D 209/42; C07C 69/24; C07D 241/04

(52) U.S. Cl. CPC C07D 209/42; C07C 69/24; C07D 241/04

(57) ABSTRACT

Methods of synthesizing (R)-N-(4-(4-(3-chloro-5-ethyl-2-methoxyphenyl) piperazin-1-yl)-3-hydroxybutyl)-1H-indole-2-carboxamide are described.

### CROSS-REFERENCE TO RELATED APPLICATIONS

This international application claims the benefit of U.S. Provisional Patent Application No. 63/325,313, filed on Mar. 30, 2022, which is incorporated by reference herein in its entirety.

### BACKGROUND

### Field

The present disclosure relates to the synthesis of (R)-N-(4-(4-(3-chloro-5-ethyl-2-methoxyphenyl) piperazin-1-yl)-3-hydroxybutyl)-1H-indole-2-carboxamide (Compound 8):

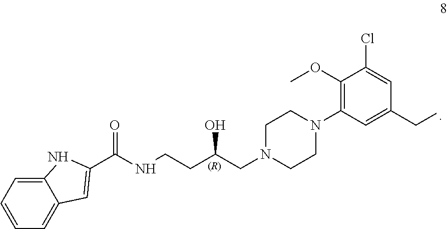

### BRIEF SUMMARY

A method of synthesizing compound 8 comprising Scheme I depicted in FIG. 1 is disclosed.

A method of synthesizing compound 8 comprising Scheme II depicted in FIG. 2 is disclosed.

A method of synthesizing compound 8 comprising Scheme III depicted in FIG. 3 is disclosed.

A method of preparing a compound 8,

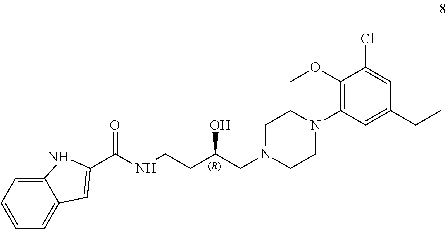

is disclosed, the method comprising:
   coupling compound 7:

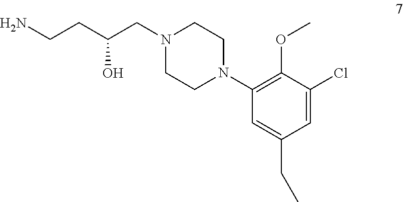 [SMILES: CCc1cc(Cl)c(OC)c(N2CCN(C[C@H](O)CCN)CC2)c1]

or a salt thereof, with 1H-indole-2-carboxylic acid in the presence of a coupling agent, wherein compound 7 is prepared by one or both of the following:
   reacting compound 6

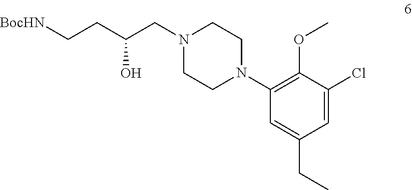 [SMILES: CCc1cc(Cl)c(OC)c(N2CCN(C[C@H](O)CCNC(=O)OC(C)(C)C)CC2)c1]

or a salt thereof, with hydrogen chloride, to provide compound 7, or a salt thereof; and
   reacting a compound 14

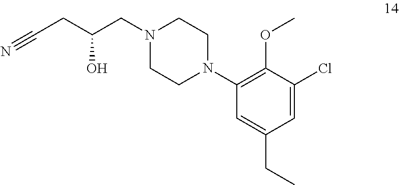 [SMILES: CCc1cc(Cl)c(OC)c(N2CCN(C[C@H](O)CC#N)CC2)c1]

or a salt thereof, with one or both of a catalyst and a reducing agent, to provide compound 7, or a salt thereof, and
wherein compound 6 is prepared by reacting compound 5:

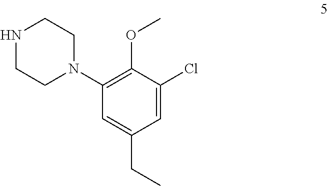 [SMILES: CCc1cc(Cl)c(OC)c(N2CCNCC2)c1]

or a salt thereof, with compound 9, Boc representing a tBuO(CO)-group:

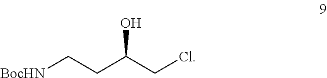 [SMILES: CC(C)(C)OC(=O)NCC[C@@H](O)CCl]

Compound 7 can be prepared by reacting a compound 6

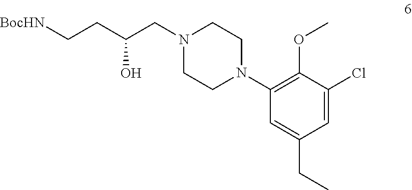 [SMILES: CCc1cc(Cl)c(OC)c(N2CCN(C[C@H](O)CCNC(=O)OC(C)(C)C)CC2)c1]

or a salt thereof, with hydrogen chloride, to provide compound 7, or a salt thereof; and
compound 6 is prepared by reacting a compound 5:

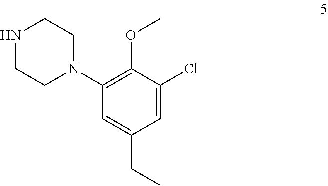 [SMILES: CCc1cc(Cl)c(OC)c(N2CCNCC2)c1]

or a salt thereof, with compound 9, Boc representing a tBuO(CO)-group:

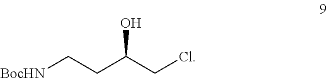 [SMILES: CC(C)(C)OC(=O)NCC[C@@H](O)CCl]

Compound 9 can be prepared by reacting (R)-4-chloro-3-hydroxybutanenitrile with Boc-anhydride and one or both of a catalyst and a reducing agent, to provide compound 9.

The reducing agent can be used for preparing compound 9.

The reducing agent can comprise lithium aluminum hydride, sodium borohydride, or hydrogen, or any combination thereof.

The catalyst can be used for preparing compound 9.

The catalyst can comprise Raney nickel, Raney cobalt, or nickel chloride, or any combination thereof.

Both the catalyst and the reducing agent can be used for preparing compound 9.

The catalyst can be nickel chloride, and the reducing agent can be sodium borohydride.

Compound 7 can be prepared by reacting compound 14

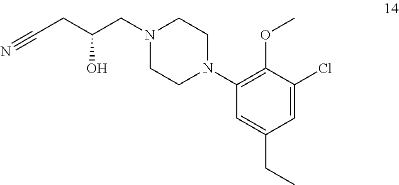 [SMILES: CCc1cc(Cl)c(OC)c(N2CCN(C[C@H](O)CC#N)CC2)c1]

or a salt thereof, with one or both of a catalyst and a reducing agent, to provide the compound 7, or a salt thereof.

The reducing agent can be used for preparing compound 7 from compound 14.

The reducing agent can comprise lithium aluminum hydride, a borane complex (for example, one or more of borane tetrahydrofuran complex, dimethyl sulfide borane, and N,N-diethylaniline borane), sodium borohydride, or hydrogen, or any combination thereof.

The catalyst can be used for preparing compound 7 from compound 14.

The catalyst can comprise Raney nickel, Raney cobalt, or nickel chloride, or any combination thereof.

Both the catalyst and the reducing agent can be used for preparing compound 14.

Compound 14 can be prepared by one or both of the following:
reacting compound 5:

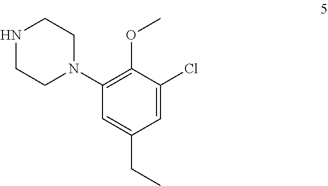 [SMILES: CCc1cc(Cl)c(OC)c(N2CCNCC2)c1]

or a salt thereof, with (S)-epichlorohydrin and cyanide or a salt thereof; and
reacting compound 5:

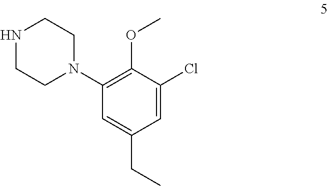 [SMILES: CCc1cc(Cl)c(OC)c(N2CCNCC2)c1]

or a salt thereof, with (R)-4-chloro-3-hydroxybutanenitrile.

Compound 14 can be prepared by reacting compound 5:

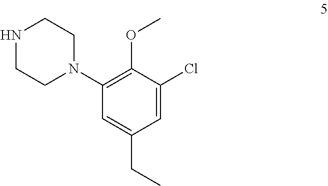 [SMILES: CCc1cc(Cl)c(OC)c(N2CCNCC2)c1]

or a salt thereof, with (S)-epichlorohydrin and cyanide or a salt thereof.

Compound 14 can be prepared by reacting compound 5:

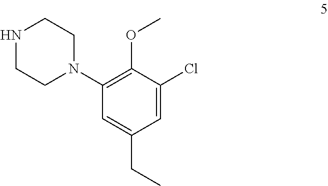 [SMILES: CCc1cc(Cl)c(OC)c(N2CCNCC2)c1]

or a salt thereof, with (R)-4-chloro-3-hydroxybutanenitrile.

Compound 5 can be prepared by reacting a compound 4:

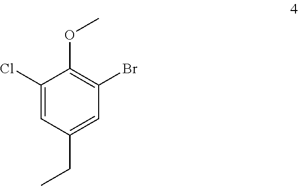

with piperazine, in the presence of a base, a diphosphine, and Pd(OAc)₂ to provide compound 5.

Compound 4 can be prepared by reacting 2-bromo-6-chloro-4-ethylphenol (3) with methyl iodide in the presence of base. [0026]2-bromo-6-chloro-4-ethylphenol (3) can be prepared by reacting 2-chloro-4-ethylphenol (2) with a brominating agent.

The brominating agent can be N-bromosuccinimide (NBS). [0028]2-chloro-4-ethylphenol (2) can be prepared by reacting 4-ethylphenol (1) with a chlorinating agent.

Compound 9

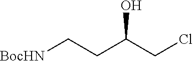 [SMILES: CC(C)(C)OC(=O)NCC[C@@H](O)CCl]

is disclosed.

A method of preparing compound 9 is disclosed by reacting (R)-4-chloro-3-hydroxybutanenitrile with Boc-anhydride and one or both of a catalyst and a reducing agent, to provide compound 9.

The reducing agent can be used for preparing compound 9.

The reducing agent can be lithium aluminum hydride, sodium borohydride, or hydrogen, or any combination thereof.

The catalyst can be used for preparing compound 9.

The catalyst can comprise Raney nickel, Raney cobalt, or nickel chloride, or any combination thereof.

Both the catalyst and the reducing agent can be used for preparing compound 9.

The catalyst can be nickel chloride, and the reducing agent can be sodium borohydride.

A method of preparing a compound of formula 11 is disclosed by reacting a compound of formula 10 with an amine of formula NHR⁴R⁵ in the presence of BINAP and palladium acetate

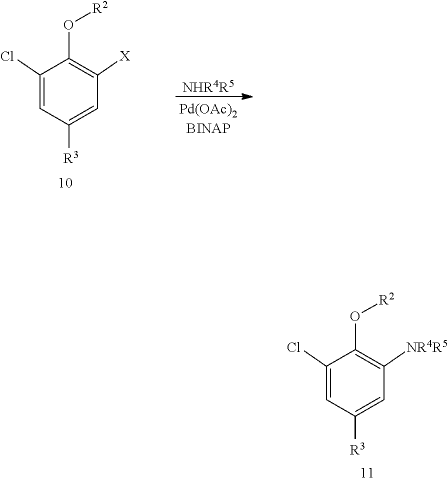

wherein
X is a halogen;
R² and R³ are independently alkyl, haloalkyl, or aryl, all of which are optionally substituted by one or more R⁶;
R⁴ and R⁵ are independently selected from H, alkyl, haloalkyl, and aryl, all of which are optionally substituted with one or more R⁶; or R⁴ and R⁵, together with the nitrogen to which they are attached, form a ring of comprising 2 to 12 carbon atoms, wherein the ring optionally comprises one or more double or triple bonds and which is ring is optionally interrupted by one or more oxygen, nitrogen or sulfur atoms;
each R⁶ is independently hydrogen, halogen, cyano, nitro, —SF₅, hydroxyl, amino, —NR⁹R¹⁰, C(O)NR⁹ R¹⁰, C₁-C₆ haloalkyl, C₁-C₆ alkyl, C₂-C₆ alkenyl, C₂-C₆ alkynyl, C₂-C₆ haloalkenyl, C₂-C₆ haloalkynyl, C₁-C₄ haloalkylsulfanyl, C₁-C₄ haloalkylsulfinyl, C₁-C₄ haloalkylsulfonyl, C₁-C₄ alkylsulfanyl, C₁-C₄ alkylsulfinyl, C₁-C₄ alkylsulfonyl, O(C₁-C₄ haloalkyl), —C(O)C₁-C₄ haloalkyl, C₁-C₆ haloalkoxy, C₁-C₆ alkoxy, or C₂-C₄ alkylcarbonyl, and
R⁹ and R¹⁰, independently from each other, are hydrogen, cyano, C₁-C₃ alkoxy or C₁-C₆ alkyl.

X can be chlorine or bromine.

X can be bromine.

R⁴ and R⁵ can comprise an N—H or NH₂ group.

The compound of formula NHR⁴R⁵ can be piperazine.

A method of preparing a compound of formula 13 is disclosed by reacting a compound 12:

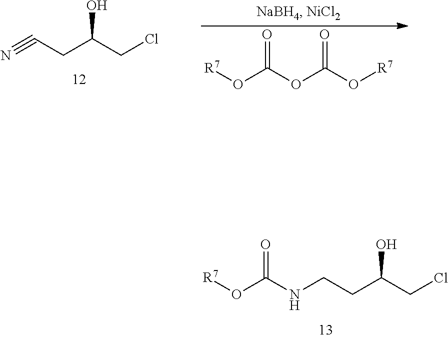

wherein the compound 12 is reacted with sodium borohydride and organic acid anhydride (15), in the presence of Ni(II) chloride, wherein R⁷ is C₁ to C₄ alkyl.

Compound 5

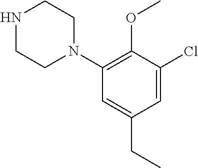 [SMILES: CCc1cc(Cl)c(OC)c(N2CCNCC2)c1]

or a salt thereof is disclosed.

Compound 6

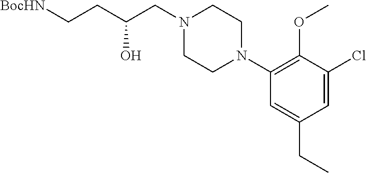 [SMILES: CCc1cc(Cl)c(OC)c(N2CCN(C[C@H](O)CCNC(=O)OC(C)(C)C)CC2)c1]

Compound 7

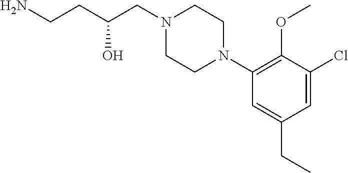 [SMILES: CCc1cc(Cl)c(OC)c(N2CCN(C[C@H](O)CCN)CC2)c1]

or a salt thereof.

A compound 7-HCl

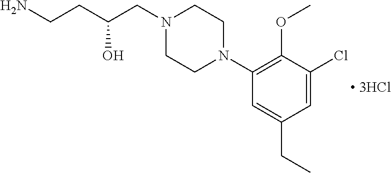 [SMILES: CCc1cc(Cl)c(OC)c(N2CCN(C[C@H](O)CCN)CC2)c1]

or a hydrate thereof is disclosed.

### Compound 14

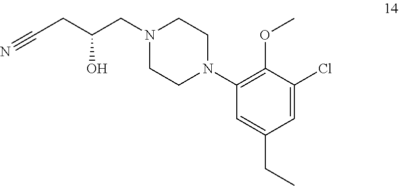 [SMILES: CCc1cc(Cl)c(OC)c(N2CCN(C[C@H](O)CC#N)CC2)c1]

or a salt thereof is disclosed.

A method of preparing a compound 8 is disclosed,

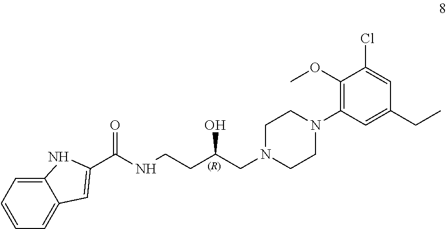

the method comprising:
a) coupling the compound 7:

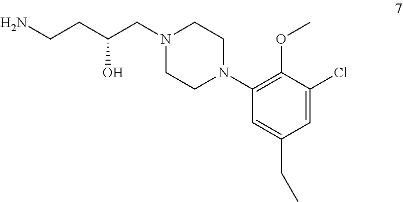 [SMILES: CCc1cc(Cl)c(OC)c(N2CCN(C[C@H](O)CCN)CC2)c1]

or a salt thereof, with 1H-indole-2-carboxylic acid in the presence of a coupling agent; and/or
b) reacting a compound 6

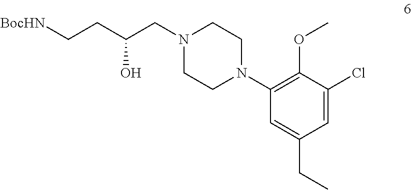 [SMILES: CCc1cc(Cl)c(OC)c(N2CCN(C[C@H](O)CCNC(=O)OC(C)(C)C)CC2)c1]

or a salt thereof, with hydrogen chloride, to provide the compound 7, or a salt thereof; and/or
c) reacting a compound 5:

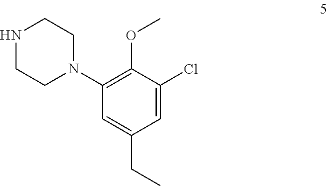 [SMILES: CCc1cc(Cl)c(OC)c(N2CCNCC2)c1]

or a salt thereof, with a compound 9

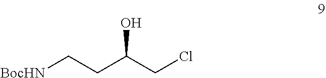 [SMILES: CC(C)(C)OC(=O)NCC[C@@H](O)CCl]

wherein Boc represents tBuO(CO)-optionally in the presence of a base; and/or
d) reacting a compound 4:

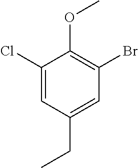

with piperazine, in the presence of a base, a diphosphine, and Pd(OAc)₂ to provide the compound 5; and/or
e) reacting 2-bromo-6-chloro-4-ethylphenol with methyl iodide in the presence of base; and/or
f) reacting 2-chloro-4-ethylphenol with a brominating agent; and/or
g) reacting 4-ethylphenol with a chlorinating agent.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 shows a synthetic route (Scheme 1) to the compound 8.

FIG. 2 shows a first synthetic route (Scheme II) to the compound 8.

FIG. 3 shows a first synthetic route (Scheme III) to the compound 8.

### DETAILED DESCRIPTION

Compounds are described using standard nomenclature. Unless defined otherwise, all technical and scientific terms used herein have the same meaning as is commonly understood by one of skill in the art to which this disclosure belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (for example, “such as”), is intended merely to better illustrate the disclosure and does not pose a limitation on the scope of the disclosure unless otherwise claimed. No language in the specification should be construed as indicating any non-claimed element as essential to the practice of the disclosure as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, for example, in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹C, ¹³C, and ¹⁴C. Accordingly, the compounds disclosed herein can include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I, and ¹³¹I. Formulae, subformulae thereof, and compounds thereof include all pharmaceutically acceptable salts of the same.

The term “substituted” means that any one or more hydrogens on the designated atom or group is replaced with a selection from the indicated group, provided that the designated atom's normal valence is not exceeded. Combinations of substituents and/or variables are permissible, for example, if such combinations result in stable compounds or useful synthetic intermediates. A stable compound or stable structure can be a compound that is sufficiently robust to survive isolation from a reaction mixture, and subsequent formulation into an effective therapeutic agent. A substituent or combination of substituents described with respect to one formula, subformula, or compound, can also be used in any other formula, subformula, or compound where consistent with valence, polarity, size, structure, and other parameters, unless otherwise indicated. Any substituent or combination of substituents described herein with respect to a particular atom or atoms can also be excluded as an option for replacing one or more hydrogens at the particular atom, atoms, or subset thereof.

“Substituted” means that the compound or group is substituted with at least one (for example, 1, 2, 3, or 4) substituent independently selected from a halogen (—F, —Cl, —Br, —I), a hydroxyl (—OH), a C₁-C₉ alkoxy, a C₁-C₉ haloalkoxy, an oxo (═O), a nitro (—NO₂), a cyano (—CN), an amino (—NR₂, wherein each R is independently hydrogen or C₁-C₁₀ alkyl), an azido (—N₃), an amidino (—C(═NH)NH₂), a hydrazino (—NHNH₂), a hydrazono (—C(═NNH₂)—), a carbonyl (—C(═O)—), a carbamoyl group (—C(O)NH₂), a sulfonyl (—S(═O)₂—), a thiol (—SH), a thiocyano (—SCN), a tosyl (CH₃C₆H₄SO₂—), a carboxylic acid (—C(═O)OH), a carboxylic C₁-C₆ alkyl ester (—C(═O)OR wherein R is C₁-C₁₀ alkyl), a carboxylic acid salt (—C(═O)OM) wherein M is an organic or inorganic anion, a sulfonic acid (—SO₃H₂), a sulfonic mono- or dibasic salt (—SO₃MH or —SO₃M₂ wherein M is an organic or inorganic anion), a phosphoric acid (—PO₃H₂), a phosphoric acid mono- or dibasic salt (—PO₃MH or —PO₃M₂ wherein M is an organic or inorganic anion), a C₁-C₁₂ alkyl, a C₃-C₁₂ cycloalkyl, a C₂-C₁₂ alkenyl, a C₅-C₁₂ cycloalkenyl, a C₂-C₁₂ alkynyl, a C₆-C₁₂ aryl, a C₇-C₁₃ arylalkylene, a C₄-C₁₂ heterocycloalkyl, and a C₃-C₁₂ heteroaryl instead of hydrogen, provided that the substituted atom's normal valence is not exceeded.

A dash (“−”) that is not between two letters or symbols is used to indicate a point of attachment for a substituent.

“Alkyl” refers to a group derived from a straight or branched chain saturated aliphatic hydrocarbon having the specified number of carbon atoms and having a valence of one, optionally substituted with one or more substituents where indicated, provided that the valence of the alkyl group is not exceeded.

“Cycloalkyl” refers to a group that comprises one or more saturated and/or partially saturated rings in which all ring members are carbon, the group having the specified number of carbon atoms. Cycloalkyl groups do not include an aromatic ring or a heterocyclic ring.

“Alkanoyl” refers to a group having formula “alkyl-C(═O)—”, wherein “alkyl” is the same as defined above.

“Cycloalkanoyl” refers to a group having formula “cycloalkyl-C(═O)—”, wherein “cycloalkyl” is the same as defined above.

“Aryl” refers to a cyclic group in which all ring members are carbon and all rings are aromatic, the group having the specified number of carbon atoms, and having a valence of one, optionally substituted with one or more substituents where indicated, provided that the valence of the aryl group is not exceeded. More than one ring can be present, and any additional rings can be fused, pendant, spirocyclic, or a combination thereof.

“Heteroaryl” means a monovalent carbocyclic ring group that includes one or more aromatic rings, in which at least one ring member (for example, one, two or three ring members) is a heteroatom selected from nitrogen (N), oxygen (O), sulfur (S), and phosphorus (P), the group having the specified number of carbon atoms.

“Halogen” means fluoro, chloro, bromo, or iodo, and are defined herein to include all isotopes of the same, including heavy isotopes and radioactive isotopes. Examples of useful halo isotopes include ¹⁸F, ⁷⁶Br, and ¹³¹I. Additional isotopes will be readily appreciated by one of skill in the art.

Compounds of formulae can contain one or more asymmetric elements such as stereogenic centers, stereogenic axes and the like, for example, asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts) of an active agent can be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers have different physical properties, for example, melting points, boiling points, spectral properties, and reactivities. Mixtures of diastereomers can separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which can occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture can occur where there has been no stereoselection or stereospecificity in a chemical reaction or process. Combinations of two enantiomeric species other than 50:50 racemic mixtures are also provided by the present disclosure, for example, 1:10,000, 1:1,000, 1:100, 1:10, 1:9, 1:7.5, 1:5, 1:3, 1:2.5, 1:2, or 1:1.5, or any opposite ratio, or any intervening ratio.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions can be carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids, for example, hydrochloric, hydrobromic, sulfuric, sulfamic, phosphoric, nitric and the like; and the salts prepared from organic acids, for example, acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Any suitable pharmaceutical salt can be used.

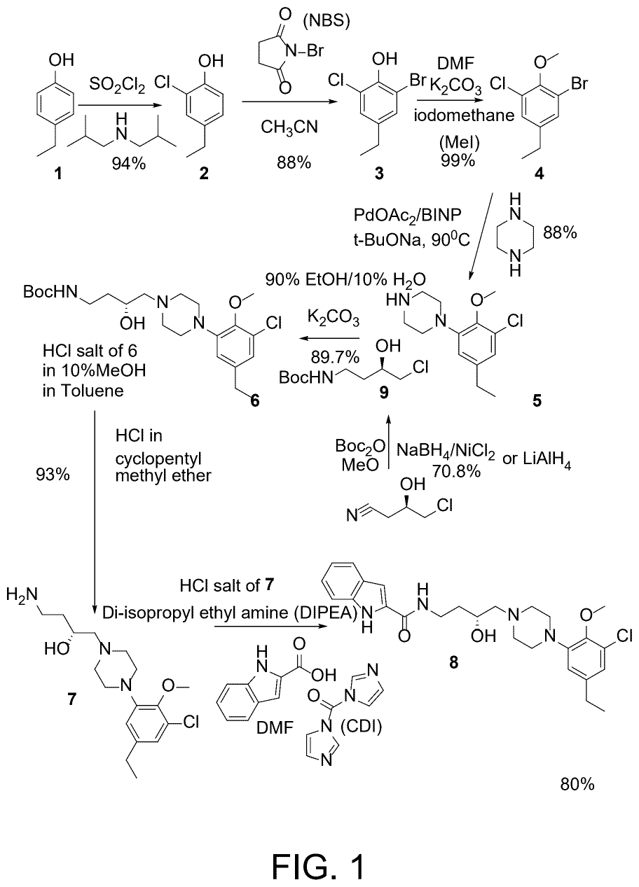

As shown in FIG. 1, 4-ethylphenol 1 can be chlorinated under conditions known to those of ordinary skill in the art, e.g., with sulfuryl chloride, to provide 2-chloro-4-ethylphenol 2. The reaction conditions can be varied according to those methods known to a person of ordinary skill in the art.

Compound 2 can be brominated to provide 2-bromo-6-chloro-4-ethylphenol 3 using N-bromosuccinimide. Compound 3 can be then methylated using methyl iodide, with an optional base to neutralize hydrogen iodide, to provide anisole 4.

The C—Br bond of compound 4 can be selectively reacted with piperazine in the presence of a palladium catalyst to provide anisole 5.

Anisole 5 can be reacted under basic conditions with chiral chlorohydrin 9, where Boc indicates tert-butoxycarbonyl, to provide alcohol 6.

The tertiary butyl carbonate (Boc) protecting group of compound 6 can be removed under acidic conditions, e.g., with hydrogen chloride, to produce amine 7 or an acid salt thereof.

Amine 7 can be condensed with 1H-indole-2-carboxylic acid using a coupling reagent to produce compound 8, or a salt thereof. Coupling agents include, for example, carbonyl diimidazole, a dialkylcarbodiimide, or a salt thereof.

Compound 9

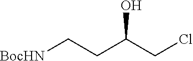 [SMILES: CC(C)(C)OC(=O)NCC[C@@H](O)CCl]

is disclosed.

A method of preparing compound 9 is disclosed by reacting (R)-4-chloro-3-hydroxybutanenitrile with Boc-anhydride and one or both of a catalyst and a reducing agent, to provide the compound 9. The reducing agent can be used for preparing compound 9. The reducing agent can be, for example, lithium aluminum hydride, sodium borohydride, or hydrogen, or any combination thereof. The hydrogen can be hydrogen gas. The catalyst can be used for preparing compound 9. Both the catalyst and the reducing agent can be used for preparing compound 9. The catalyst can be, for example, nickel chloride, and the reducing agent can be, for example, sodium borohydride. Reducing agents, catalysts, and the like described for producing compound 9 can be used for producing compound 7 from compound 14 and vice versa. Choice of reducing agents, catalysts, and the like can affect yield.

Compound 9 can be used in the synthesis of pharmaceutical compounds and salts thereof, for example, dopamine receptor-targeted compounds such as compound 8.

Compound 9 can also be used to synthesize other classes of drugs, for example, statins such as atorvastatin, pravastatin, pitavastatin, rosuvastatin, fluvastatin, or cerivastatin, or any combination thereof.

A method of synthesizing the compound of formula 11 from a compound of formula 10 is disclosed:

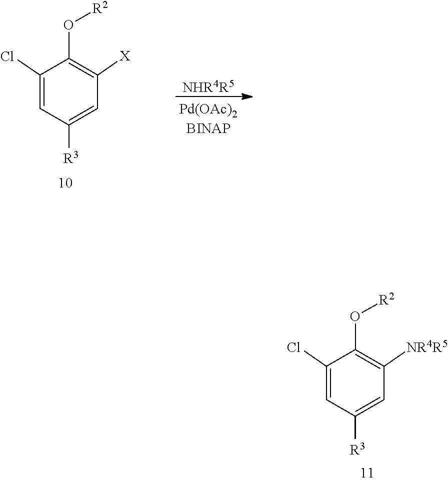

wherein:
X is a halogen;
R² and R³ are independently alkyl, haloalkyl, or aryl, all of which are optionally substituted by one or more R⁶;
R⁴ and R⁵ are independently selected from H, alkyl, haloalkyl, and aryl, all of which are optionally substituted with one or more R⁶; or R⁴ and R⁵, together with the nitrogen to which they are attached, form a ring of comprising 2 to 12 carbon atoms, wherein the ring optionally comprises one or more double or triple bonds and which is ring is optionally interrupted by one or more oxygen, nitrogen or sulfur atoms.
Each R⁶ is independently hydrogen, halogen, cyano, nitro, —SF₅, hydroxyl, amino, —NR⁹R¹⁰, C(O)NR⁹ R¹⁰, C₁-C₆ haloalkyl, C₁-C₆ alkyl, C₂-C₆ alkenyl, C₂-C₆ alkynyl, C₂-C₆ haloalkenyl, C₂-C₆ haloalkynyl, C₁-C₄ haloalkylsulfanyl, C₁-C₄ haloalkylsulfinyl, C₁-C₄ haloalkylsulfonyl, C₁-C₄ alkylsulfanyl, C₁-C₄ alkylsulfinyl, C₁-C₄ alkylsulfonyl, O(C₁-C₄ haloalkyl), —C(O)C₁-C₄ haloalkyl, C₁-C₆ haloalkoxy, C₁-C₆ alkoxy, or C₂-C₄ alkylcarbonyl.
R⁹ and R¹⁰, independently from each other, are hydrogen, cyano, C₁-C₃ alkoxy or C₁-C₆ alkyl.

R⁴ and R⁵ can be substituted with an N—H, or NH₂ group.

The reaction can be carried out in a solvent, for example a non-polar solvent. Typical solvents include alkanes or aryl solvents. For example, some non-halogenated solvents, e.g., aromatic solvents, can be used. Such solvents include, for example, benzene, toluene, o-xylene, m-xylene, p-xylene, or chlorobenzene or combinations thereof.

The molar ratio of the amine HNR⁴R⁵ to the compound 10 can be from 10:1 to 2:1, from 9:1 to 2:1, from 8:1 to 2:1, from 7:1 to 2:1, from 6:1 to 2:1, from 5:1 to 2:1, from 4:1 to 2:1, from 3:1 to 2:1, from 8:1 to 3:1, or from 6:1 to 5:1.

The reaction can be carried out in the presence of a base, for example, a tertiary amine base (e.g., triethylamine), or an alkoxide base, e.g., a tert-butoxide, an iso-propoxide, an ethoxide, and the like. In an embodiment, the molar ratio of the base to the compound 10 can be from 10:1 to 1:1, from 9:1 to 1:1, from 8:1 to 1:1, from 7:1 to 1:1, from 6:1 to 1:1, from 5:1 to 1:1, from 4:1 to 1:1, from 3:1 to 1:1, or from 2:1 to 1:1.

The ligand known as BINAP can used in the reaction. BINAP is an acronym or abbreviation of the name ([1,1′-Binaphthalene]-2,2′-diyl)bis(diphenylphosphane).

Other phosphorous ligands can be used as well, for example those known to the person of ordinary skill in the art, including but not limited to: 1,1-Bis(diphenylphosphino)methane (dppm); 1,2-Bis(dimethylphosphino)ethane (dmpe); 1,2-Bis(diisopropylphosphino)ethane (dippe); 1,2-Bis(diphenylphosphino)benzene (dppbz); 1,2-Bis(diphenylphosphino)ethane (dppe); derivative of phenylanisylmethylphosphine (DIPAMP); Bis(dicyclohexylphosphino)ethane (dcpe); 1,3-Bis(diphenylphosphino)propane (dppp); 1,4-Bis(diphenylphosphino)butane (dppb); (S,S)-DIOP (0-isopropylidene-2,3-dihydroxy-1,4-bis(diphenylphosphino)butane) (DIOP); 2,3-Bis(diphenylphosphino)butane (Chiraphos); 4,5-Bis(diphenylphosphino)-9,9-dimethylxanthene (Xantphos); Bis[(2-diphenylphosphino)phenyl]ether (DPEphos); 4,4,4′,4′,6,6′-Hexamethyl-2,2′-spirobichromane-8,8′-diylbis(diphenylphosphane) (SPANphos); 4,4′-Bi-1,3-benzodioxole-5,5′-diylbis(diphenylphosphane) (SEGPHOS); 1,1′-Bis(diphenylphosphino)ferrocene (dppf); 1,2-Bis(2,5-dimethylphospholano)benzene (Me-DuPhos); (Diphenylphosphino)ferrocenyl-ethyldicyclohexylphosphine1,5-Diaza-3,7-diphosphacyclooctanes (Josiphos); and 1,5-Diaza-3,7-diphosphacyclooctanes (P2N2).

Palladium (II) acetate and the diphosphine ligand, e.g., BINAP, can be used in catalytic amounts, e.g., from 0.0001 to 0.01 molar equivalents, each amount selected independently of one another. In an embodiment, the amounts of Palladium (II) acetate and diphosphine ligand used are, independently, 0.0001, 0.0005, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, or 0.01 molar equivalents.

A method of synthesizing a compound of formula 13 from a compound of formula 12 is disclosed:

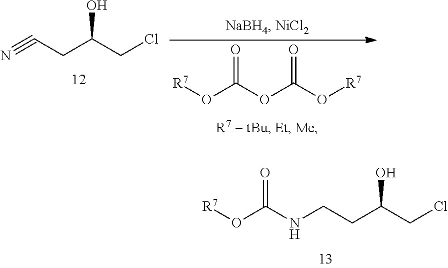

R⁷ can be from C₁ to C₄ alkyl, for example, tert-butyl, isobutyl, sec-butyl, n-butyl, n-propyl, isopropyl, ethyl or methyl. The compound 12, (R)-4-Chloro-3-hydroxybutyronitrile, can be reacted with a Lewis acid and a borohydride in the presence of a dialkyl decarbonate, e.g., di-tert-butyldicarbonate, also known as Boc anhydride.

The borohydride, which functions as a reducing agent of the nitrile in association with the Lewis acid, can be a conventional borohydride, e.g., sodium bororhydride, lithium borohydride, sodium triacetoxyborohydride, tetrabutylammonium borohydride, or another borohydride known to a person of ordinary skill in the art. In an embodiment, the molar ratio of the borohydride to the nitrile is from about 3:1 to about 1.1:1, from about 2.9:1 to about 1.1:1, from about 2.8:1 to about 1.1:1, from about 2.7:1 to about 1.1:1, from about 2.6:1 to about 1.1:1, from about 2.5:1 to about 1.1:1, from about 2.4:1 to about 1.1:1, from about 2.3:1 to about 1.1:1, from about 2.2:1 to about 1.1:1, from about 2.1:1 to about 1.1:1, from about 2:1 to about 1.1:1, from about 1.9:1 to about 1.1:1, from about 1.8:1 to about 1.1:1, from about 1.7:1 to about 1.1:1, from about 1.6:1 to about 1.1:1, from about 1.5:1 to about 1.1:1, from about 1.4:1 to about 1.1:1, from about 1.3:1 to about 1.1:1, or from about 1.2:1 to about 1.1:1.

The Lewis acid associated with the borohydride can comprise a Group I (alkali) metal ion, Group II (alkaline earth) metal ion, Group III non-metal or metal atom, molecule, or ion, and/or a Row 3 Transition metal ion, e.g., Ti, V, Cr, Mn, Fe, Co, Ni, Cu, and/or Zn. For example, such molecules comprising Row 3 metal ions include TiCl₄, FeCl₃, CuCl₂, CuCl, NiCl₂, ZnCl₂, and the like, as known to the person of ordinary skill in the art. In an embodiment, the molar ratio of the Lewis acid to the nitrile is from about 0.01:1 to about 0.9:1, from about 0.05:1 to about 0.9:1, from about 0.1:1 to about 0.9:1, from about 0.15:1 to about 0.9:1, from about 0.2:1 to about 0.9:1, from about 0.25:1 to about 0.9:1, from about 0.3:1 to about 0.9:1, from about 0.35:1 to about 0.9:1, from about 0.4:1 to about 0.9:1, from about 0.45:1 to about 0.9:1, from about 0.5:1 to about 0.9:1, from about 0.55:1 to about 0.9:1, from about 0.6:1 to about 0.9:1, from about 0.65:1 to about 0.9:1, from about 0.7:1 to about 0.9:1, from about 0.75:1 to about 0.9:1, from about 0.8:1 to about 0.9:1, or from about 0.85:1 to about 0.9:1, from about 0.1:1 to about 0.5:1, from about 0.15:1 to about 0.5:1, from about 0.2:1 to about 0.5:1, from about 0.25:1 to about 0.5:1, from about 0.3:1 to about 0.5:1, from about 0.35:1 to about 0.5:1, from about 0.4:1 to about 0.5:1, or from about 0.45:1 to about 0.5:1, or from about 0.1:1 to about 0.5:1.

The reaction can be carried out in a solvent, for example, a hydroxylic solvent, such as an alcohol. Typical solvents include alkyl alcohols such as propanol, isopropanol, ethanol, and methanol. The reaction can be carried out under anhydrous conditions.

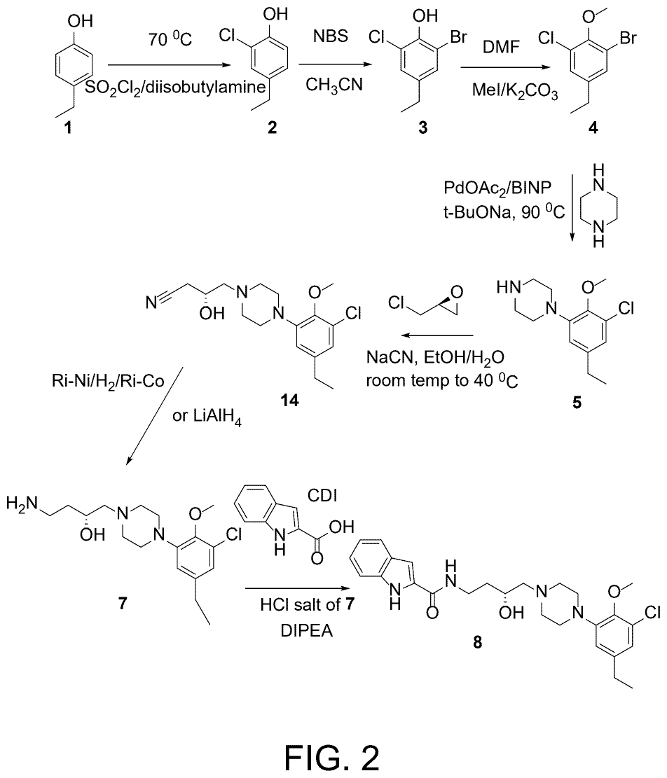

The present disclosure also provides a second route to compound 8 in which the intermediate between compounds 5 and 7 is compound 14 instead of compound 6. This second route (Scheme II) is depicted in FIG. 2.

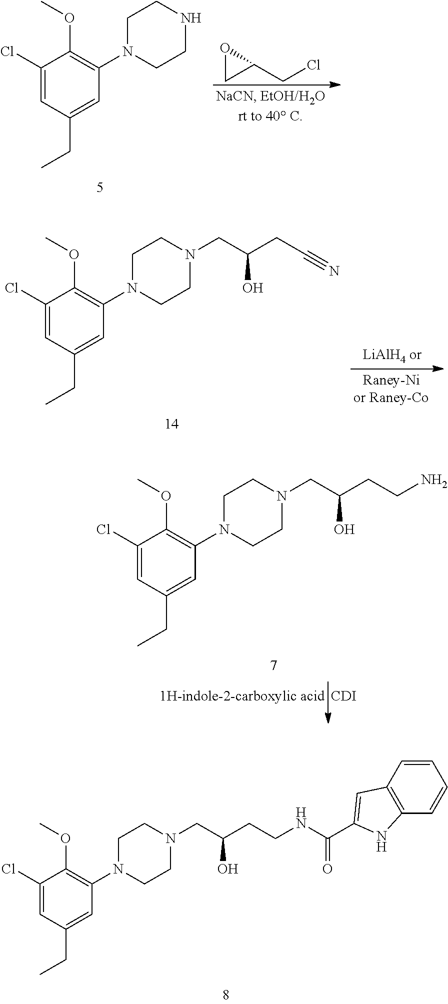

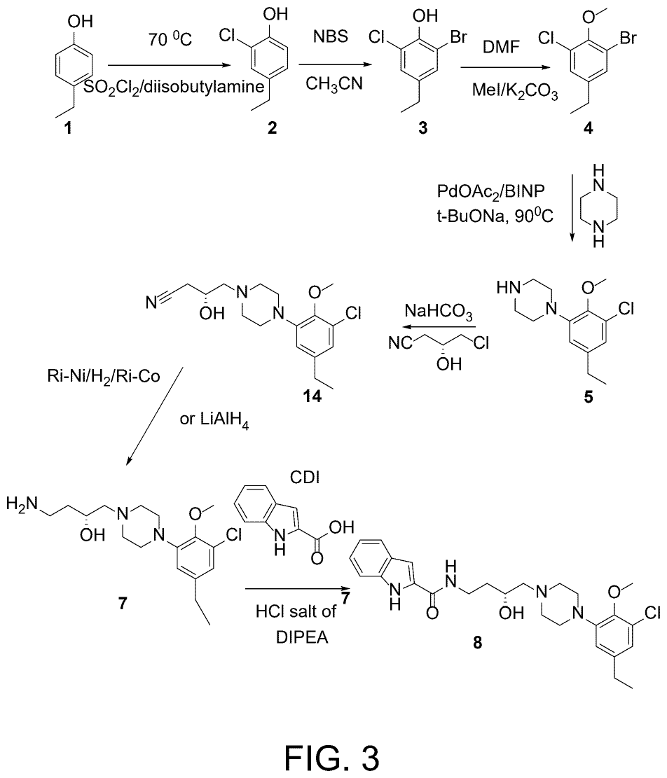

The present disclosure further provides a third route to compound 8 in which the intermediate is compound 14 instead of compound 6 as in Scheme II, but a different reagent is employed to form compound 14. This third route (Scheme III) is depicted in FIG. 3. For Schemes II and III, alternative or additional reducing agents and/or catalysts can be used, for example, those described in Scheme I, as well as one or more borane complexes, for example, borane tetrahydrofuran complex, dimethyl sulfide borane, or N,N-diethylaniline borane, or any combination thereof.

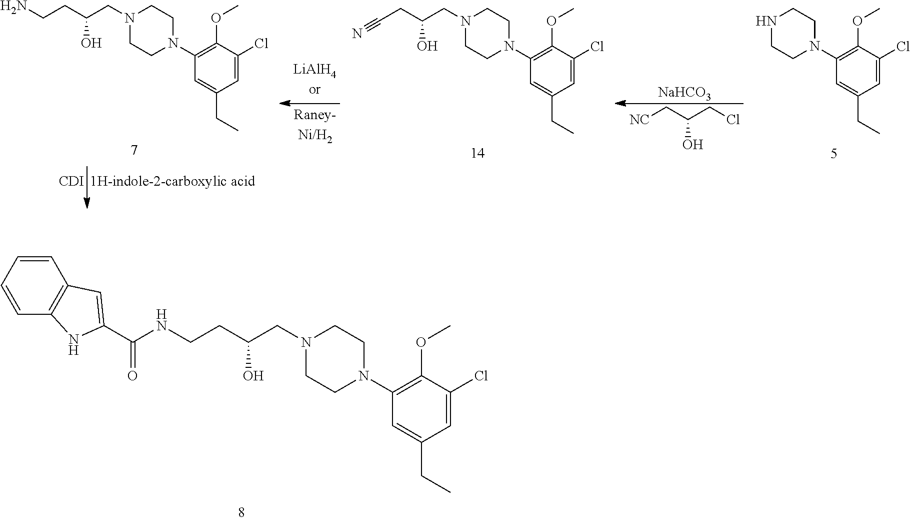

### EXAMPLES

### Example 1

This example sets forth reaction conditions for the first step of Schemes I-III in which compound 2 is formed from compound 2.

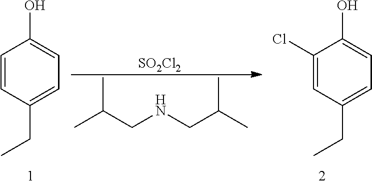

Sulfuryl chloride (146 mL, 1.801 mol) in 600 mL toluene was added dropwise during 30 min into a solution of 4-ethylphenol (Compound 1, 200 g, 1.64 mol) and di-isobutylamine (28.6 mL, 0.164 mol) in toluene (1.2 L) at 70° C. This reaction mixture was heated at 70° C. for 1 hour and cooled to room temperature and diluted with 2 L of methyl tert-butyl ether. The mixture was transferred into 1 kg ice and 1 L water in a 12 L workup reactor. The resulting mixture was stirred at room temperature for 30 min. and the aqueous layer was discarded after GC-MS indicated no product remained in it. The combined organic phase was washed with brine (2×500 mL) and dried over Na₂SO₄. The solvent was removed under reduced pressure (15 torr/40° C.) to obtain compound 2, 2-chloro-4-ethylphenol, (260 g, 93% pure/GC, 94% yield) as a yellowish oil.

### Example 2

This example sets forth reaction conditions for the second step of Schemes I-III in which compound 3 is formed from compound 2.

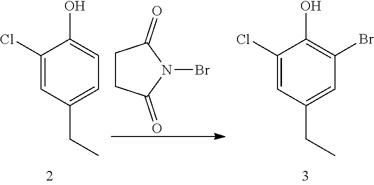

N-bromosuccinimide (275 g, 1.544 mol) was added portion-wise to a solution of compound 2 (2-chloro-4-ethylphenol, 260 g, 1.544 mol) in acetonitrile (1.56 L) below 35° C. with water cooling. A slight exotherm was observed. The mixture was stirred at room temperature overnight (20 hours) and then diluted with water (1000 mL). The resulting mixture was extracted with 5×500 mL hexanes. Combined organic phase was washed first with 500 mL brine, Na₂S₂O₃ (saturated aqueous solution) 2×200 mL and brine 3×500 mL. The combined organic layer was dried over Na₂SO₄ (50 g). Solvent was evaporated under reduced pressure (15 torr/40° C.) to obtain 2-bromo-6-chloro-4-ethylphenol 3 (320 g, 88%) as a crude brown oil that was used without further purification.

### Example 3

This example sets forth reaction conditions for the third step of Schemes I-III in which compound 4 is formed from compound 3.

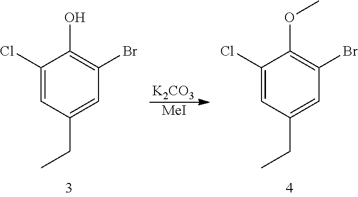

Potassium carbonate (282 g, 2.038 mol) and iodomethane (127 mL, 2.038 mol) were added to a solution of 2-bromo-6-chloro-4-ethylphenol (Compound 3, 320 g, 1.359 mol) in N,N-dimethylformamide (1440 mL) at room temperature under N₂. This mixture was heated to 70° C. for 4 hours. The reaction was cooled to room temperature and diluted with 3 L water. This mixture was extracted with hexanes (4×500 mL) and the combined organic phase was washed with brine (3×500 mL) and dried over Na₂SO₄ (50 g) and solvent was removed under reduced pressure (15 torr/40° C.) to obtain 2-bromo-6-chloro-4-ethylanisole 4 (336 g, 99% yield). ¹H NMR (400 MHz, CDCl₃) δ 7.28 (dd, J=2.1, 0.7 Hz, 1H), 7.16 (dd, J=2.0, 0.7 Hz, 1H), 3.87 (s, 3H), 2.57 (q, J=7.5 Hz, 2H); 1.21 (t, J=7.6 Hz, 3H)¹³C NMR (101 MHz, CDCl₃) δ 151.22, 142.43, 131.34, 129.19, 128.83, 118.20, 60.78, 28.00, 15.38.

### Example 4

This example sets forth reaction conditions for the fourth step of Schemes I-III in which compound 5 is formed from compound 4.

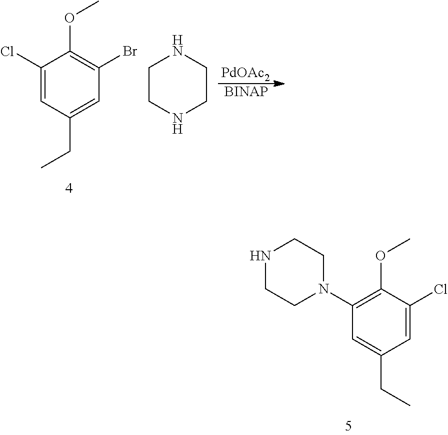

Piperazine (414 g, 4.809 mol, 6 eq) and tert-BuONa (139 g, 1.443 mol, 1.8 eq) were added to a solution of 2-bromo-6-chloro-4-ethylanisole 4 (200 g, 0.802 mol, 1 eq) in 1000 mL toluene at room temperature with N₂ inlet and mechanical stirring. This mixture was degassed for 10 min. via bubbling N₂. 2,2′-bis(diphenylphosphino)-1,1′-binaphthyl (BINAP) (2.99 g, 4.81 mmol, 0.6% eq) and Pd(OAc)₂ (0.344 g, 1.603 mmol, 0.2% eq) were added and the resulting mixture degassed for another five minutes. The mixture was heated to 90° C. overnight with small stream of N₂ bubbling in during the whole process to keep 02 off. The reaction mixture was cooled to room temperature, and subsequently diluted with toluene (1 L) and methyl tert-butyl ether (1 L). The mixture was filtered through Celite and washed with methyl tert-butyl ether (3×500 mL). The combined organic phase was washed with 4×500 mL brine and dried over Na₂SO₄ for three hours. The dried organic phase was transferred to a 5 L reactor and 3M HCl in cyclopentyl methyl ether (267 mL, 0.802 mol, 1 eq) was added dropwise at room temperature. The resulting mixture was stirred at room temperature for one hour. A precipitate was filtered and washed with methyl tert-butyl ether (3×500 mL) and dried over high vacuum to obtain 205 g off white solid (88% yield) of compound 5. ¹H NMR (400 MHz, CDCl₃) δ 9.94 (s, 2H), 6.94 (d, J=1.9 Hz, 1H), 6.70 (d, J=2.0 Hz, 1H), 3.83 (s, 3H), 3.47 (q, J=5.7 Hz, 9H), 2.53 (q, J=7.6 Hz, 2H), 1.18 (t, J=7.6 Hz, 3H); ¹³C NMR (101 MHz, CDCl₃) δ 146.85, 144.21, 141.53, 128.80, 124.70, 117.47, 60.02, 47.85, 43.96, 28.54, 15.48.

### Example 5

This example sets forth reaction conditions for forming compound 9 from compound 12 as a reactant for the fifth step of Scheme I.

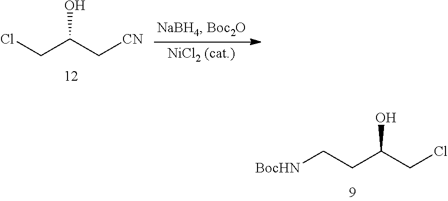 [SMILES: N#CC[C@@H](O)CCl ; CC(C)(C)OC(=O)NCC[C@@H](O)CCl]

Sodium borohydride (27.7 g, 2.0 eq) was added portion-wise to a mixture of (R)-4-chloro-3-hydroxybutanenitrile 12 (43 g, 41.8 mmol), Boc-anhydride (1.2 eq) and NiCl₂ (0.1 eq) in MeOH (800 mL) with ice-salt bath cooling (−10 to −15° C. outside) to maintain an internal reaction temperature below 17° C. The reaction was stirred at room temperature overnight. GCMS analysis of the reaction mixture showed about 6-7% of compound 12 remaining. This mixture was cooled again with ice-water bath, and more NaBH₄ was added (3.8 g). The reaction was stirred at room temperature for another 6 h until GC-MS showed no starting material. The reaction was cooled to 5° C. and 1 N HCl solution was added (˜850 mL) to reach pH 3. The mixture was extracted with methyl tert-butyl ether, and washed with 0.3 N HCl, water, brine, dried and concentrated to give tert-butyl (R)-(4-chloro-3-hydroxybutyl) carbamate (57 g, 255 mmol, 70.8% yield) as light-yellow oil. The crude material was treated with imidazole (0.4 eq) in ethanol, which was diluted with water and extracted with MTBE. After removal of solvent tert-butyl (R)-(4-chloro-3-hydroxybutyl) carbamate 9, 96.4 wt % pure, was isolated. ¹H NMR (400 MHz, Chloroform-d) δ 4.90 (s, 1H), 3.87 (dq, J=10.0, 5.0, 4.1 Hz, 1H), 3.62-3.39 (m, 3H), 3.18 (dq, J=14.6, 5.4 Hz, 1H), 1.84-1.68 (m, 1H), 1.66-1.54 (m, 1H), 1.44 (s, 9H), ¹³C NMR (101 MHz, Chloroform-d) δ 156.90, 79.75, 69.04, 49.56, 37.07, 34.78, 28.36.

### Alternative Example 5

To a 12 L 4-neck flask equipped with mechanic stirrer, reflux condenser, (R)-4-chloro-3-hydroxybutanenitrile (200 g, 1.673 mol, 1.0 eq) was added, followed by methanol (4000 ml), Boc-anhydride (548 ml, 2.51 mol, 1.5 eq) and nickel (II) chloride (19.51 g, 151 mmol, 0.09 eq). The reaction mixture was cooled to 0° C. (outside −10 to −15° C.), and then NaBH₄ (190 g, 5.019 mol, 3.0 eq) was added by portions in order to maintain an internal temperature below 5° C. (Typically, the procedure comprised observed H₂ gas release; 7-8 g of NaBH₄ constituted a portion; after each portion was added, 10-15 min passed until the intern temperature cooled to 0° C. again). The entire amount of NaBH₄ was added over 3.5 h. The reaction was allowed to warm to room temperature and stirred at 22° C. over a weekend; reaction progress was followed with GCMS. The reaction was cooled with ice-bath to 0° C., diluted with 1.5 L of methyl tert-butyl ether; and 20% of citric acid solution was added (1000 mL) until pH=4.05. Methyl tert-butyl ether (850 mL) was added to the mixture, which was transferred to a 12 L separation reactor followed by the addition of water (2 L). The organic layer was separated, and aqueous layer was extracted with methyl tert-butyl ether (800 mL×2). The combined organic layer was washed with 20% citric acid (500 mL), water (500 mL), saturated NaHCO₃ in water solution (500 mL), brine (500 mL), dried with Na₂SO₄, filtered, and concentrated under reduced pressure at 40° C. to give crude tert-butyl (R)-(4-chloro-3-hydroxybutyl)-carbamate as a light-yellow oil. The crude material was treated with imidazole (45.6 g, 0.669 mol, 0.4 eq) in 10% ethanol in methyl tert-butyl ether (2 L) and it was stirred at room temperature for 3 hours. the reaction mixture was washed with brine (3×300 mL). The organic layer was dried over Na₂SO₄ and solvent was removed under reduce pressure to obtain tert-butyl (R)-(4-chloro-3-hydroxybutyl) carbamate 9, 96.4 wt % pure (195 g, 52.1%).

### Example 6

This example sets forth reaction conditions for the fifth step of Scheme I in which compound 6 is formed from compound 5.

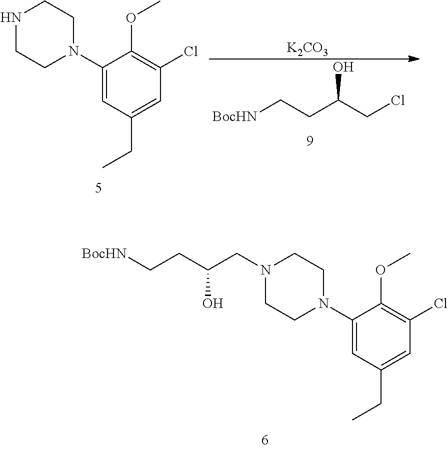 [SMILES: CCc1cc(Cl)c(OC)c(N2CCNCC2)c1 ; "K2CO3" ; CC(C)(C)OC(=O)NCC[C@@H](O)CCl ; CCc1cc(Cl)c(OC)c(N2CCN(C[C@H](O)CCNC(=O)OC(C)(C)C)CC2)c1]

Potassium carbonate (350 g, 2532 mmol, 2.5 eq) was added portion-wise to a mixture of the hydrochloride salt of 1-(3-chloro-5-ethyl-2-methoxyphenyl)-piperazine 5 (295 g, 1013 mmol, 1 eq) in 90% EtOH/10% water solution (3000 mL) at room temperature under N₂ and the reaction stirred at room temperature for 30 min. Then tert-butyl (R)-(4-chloro-3-hydroxybutyl) carbamate 9 (378 g, 1519 mmol, 1.5 eq, 90% wt) was added. This mixture was heated to reflux overnight. The reaction vessel was cooled to room temperature and diluted with 6 L methyl tert-butyl ether and 6 L water. The organic layer was washed with brine 3×1000 mL, dried over Na₂SO₄ and all solvent was removed by reduced pressure (40° C., 15 torr) and the remaining brown oil was placed under high vacuum (0.5 torr) overnight to provide 578.89 g of a brown oil (129%) containing crude compound 6.

### Example 7

This example sets forth reaction conditions for the formation of the hydrochloride salt of compound 6 as a reactant in the sixth step of Scheme I.

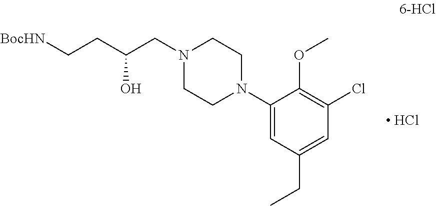 [SMILES: CCc1cc(Cl)c(OC)c(N2CCN(C[C@H](O)CCNC(=O)OC(C)(C)C)CC2)c1 ; "· HCl"]

3N HCl in cyclopentyl methyl ether (437 mL, 1310 mmol, 1 eq) was added dropwise over 42 minutes to a solution of tert-butyl (R)-(4-(4-(3-chloro-5-ethyl-2-methoxyphenyl)-piperazin-1-yl)-3-hydroxybutyl) carbamate 6 (crude, 579 g, 1310 mmol, 1 eq) in methyl tert-butyl ether (5800 mL) at 2-5° C. The reaction was stirred at this temperature for 30 min. The resulting precipitate was filtered under N₂ and solid was washed with 3×1000 mL methyl tert-butyl ether, and the collected solid was slurried in 4000 mL of methyl tert-butyl ether for 3 hours, filtered under N₂ again and dried under high vacuum to obtain 435 g (69.4%) of 6-HCl as an off-white solid.

### Example 8

This example sets forth reaction conditions for the sixth step of Scheme I in which compound 7 is formed from the hydrochloride salt of compound 6.

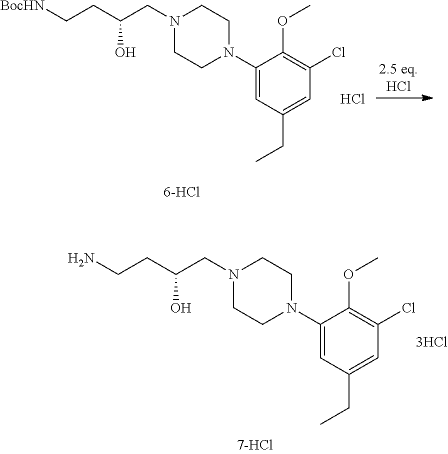 [SMILES: CCc1cc(Cl)c(OC)c(N2CCN(C[C@H](O)CCNC(=O)OC(C)(C)C)CC2)c1 ; Cl ; CCc1cc(Cl)c(OC)c(N2CCN(C[C@H](O)CCN)CC2)c1 ; "3HCl"]

The HCl salt of tert-butyl (R)-(4-(4-(3-chloro-5-ethyl-2-methoxyphenyl)-piperazin-1-yl)-3-hydroxybutyl) carbamate 6-HCl (427 g, 171 mmol, 1 eq) was dissolved in 4270 mL 10% MeOH in toluene (10 vol), and 3 M HCl (744 mL, 2231 mmol, 2.5 eq) in cyclopentyl methyl ether was added dropwise at room temperature during 40 min. This mixture was stirred at room temperature overnight. A precipitate was filtered under N₂ after LCMS showed tert-butyl (R)-(4-(4-(3-chloro-5-ethyl-2-methoxyphenyl) piperazin-1-yl)-3-hydroxybutyl) carbamate 6-HCl had disappeared. The solid was washed with 10% MeOH in toluene (2×1000 mL) and then methyl tert-butyl ether (3×1000 mL). Then the resulting crude solid was slurried in 10% MeOH in methyl tert-butyl ether (4 L) overnight. A precipitate was filtered and washed with 2×1000 mL 10% methanol in methyl tert-butyl ether and 3×1000 mL methyl tert-butyl ether. The solid was filtered under N₂ overnight. The grey solid was dried under high vacuum to obtain 373 g of 7-HCl (R)-4-amino-1-(4-(3-chloro-5-ethyl-2-methoxyphenyl)-piperazin-1-yl)-butan-2-ol, 3-HCl (93%). ¹H NMR (400 MHz, DMSO-d6) δ 10.64 (s, 1H), 8.19 (d, J=5.9 Hz, 3H), 6.94 (d, J=1.8 Hz, 1H), 6.77 (d, J=2.0 Hz, 1H), 4.21 (q, J=8.7 Hz, 1H), 3.58 (q, J=12.5, 11.2 Hz, 5H), 3.34-3.11 (m, 6H), 3.14-3.02 (m, 1H), 2.96-2.78 (m, 3H), 2.57-2.45 (m, 3H), 1.80-1.60 (m, 2H), 1.13 (t, J=7.6 Hz, 3H). ¹³C NMR (101 MHz, DMSO-d6) δ 146.12, 144.92, 141.51, 127.72, 122.97, 117.81, 62.52, 61.24, 59.42, 53.03, 51.43, 46.87, 46.80, 36.05, 32.83, 28.07, 15.89.

### Example 9

This example sets forth reaction conditions for the seventh step of Schemes I-III in which compound 8 is formed from compound 7.

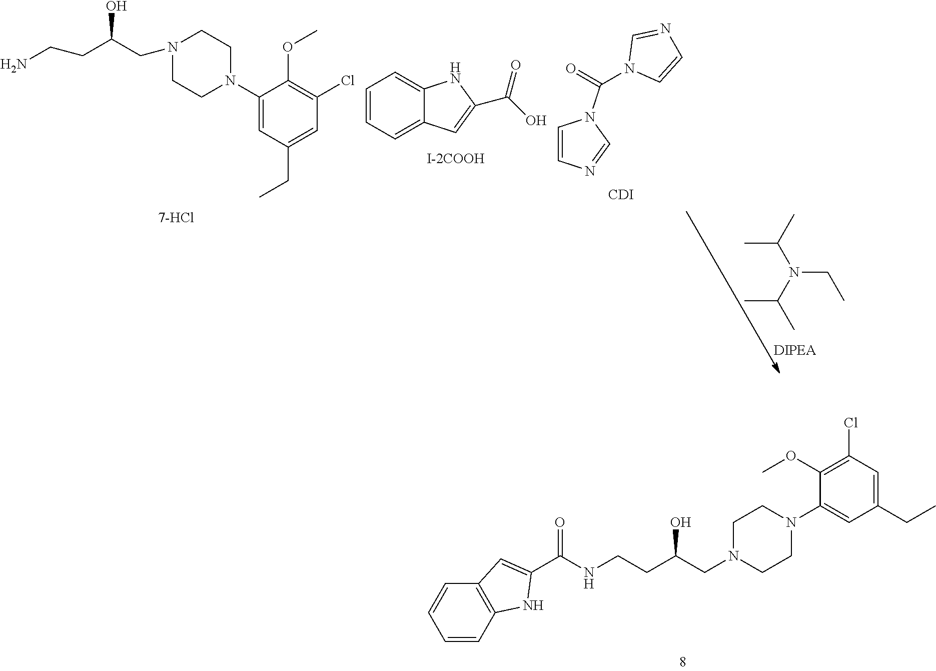

A mixture of carbonyl diimidazole (CDI, 154 g, 951 mmol, 1.15 eq) and 1H-indole-2-carboxylic acid (I-2COOH, 153 g, 951 mmol, 1.15 eq) in 1800 mL anhydrous N,N-dimethyl formamide (9 L) was stirred at room temperature for 3 h. Di-isopropyl ethyl amine (DIPEA, 589 mL, 3306 mmol, 4 eq) was added to the mixture, followed by (R)-4-amino-1-(4-(3-chloro-5-ethyl-2-methoxyphenyl)-piperazin-1-yl)-butan-2-ol, 3HCl (7-HCl, 373 g, 827 mmol, 1 eq) portion-wise over 5 min. at room temperature under N₂. A slight exotherm was observed. The mixture was stirred at room temperature overnight. Water (1800 mL) was added dropwise over 45 min, and solid was precipitated and it was stirred for an additional 30 min., then the precipitate was filtered. The precipitate was washed with 2×1000 mL 50% N,N-dimethyl formamide in water, and 4×1000 mL water. The crude wet solid was added to 3600 mL MeOH and treated with 276 mL 3 N NaOH (827 mmol, 1 eq). The resulting mixture was stirred at room temperature for 3 hours. Water (3600 mL) was added dropwise over 50 minutes and the mixture stirred for further 30 min, then the resulting precipitate was filtered, and washed with 50% MeOH in water (2×1000 mL) and water (4×1000 mL). The precipitate was filtered and dried under N₂ overnight using a Buchner filtration system. The solid was then dried under high vacuum overnight to obtain (R)—N-(4-(4-(3-chloro-5-ethyl-2-methoxyphenyl)-piperazin-1-yl)-3-hydroxybutyl)-1H-indole-2-carboxamide 8 (322 g, 80%) as a grey solid with 98.87% (290 nm) purity. ¹H NMR (400 MHz, DMSO-d6) δ 11.52 (d, J=2.2 Hz, 1H), 8.43 (t, J=5.7 Hz, 1H), 7.58 (d, J=8.0 Hz, 1H), 7.40 (d, J=8.2 Hz, 1H), 7.18-7.10 (m, 1H), 7.10-7.05 (m, 1H), 7.00 (dd, J=7.9, 6.7 Hz, 1H), 6.85 (d, J=1.9 Hz, 1H), 6.67 (d, J=2.0 Hz, 1H), 4.48 (d, J=4.5 Hz, 1H), 3.78-3.71 (m, 1H), 3.71 (s, 3H), 3.39 (ddq, J=37.9, 12.7, 7.1, 6.2 Hz, 2H), 3.01 (t, J=4.9 Hz, 4H), 2.57 (t, J=4.7 Hz, 4H), 2.54-2.43 (m, 3H), 2.34 (qd, J=12.5, 6.2 Hz, 2H), 1.79 (dtd, J=16.0, 7.7, 3.3 Hz, 1H), 1.59-1.46 (m, 1H), 1.11 (t, J=7.6 Hz, 3H). ¹³C NMR (101 MHz, DMSO-d6) δ 161.57, 146.14, 144.92, 141.49, 136.83, 132.28, 127.72, 127.49, 123.64, 122.96, 121.85, 120.11, 117.81, 112.70, 103.29, 62.88, 61.38, 59.42, 53.12, 51.38, 46.93, 46.84, 35.56, 35.31, 28.08, 15.88.

### Example 10

This example sets forth reaction conditions for the fifth step of Scheme II in which compound 14 is formed from compound 5.

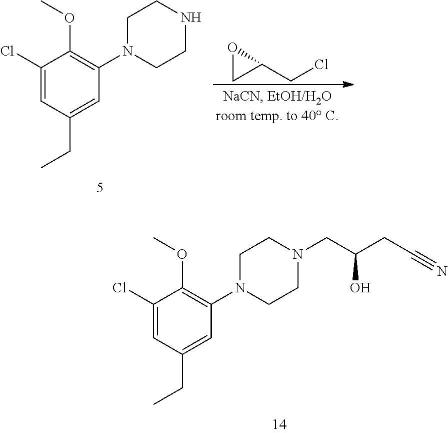 [SMILES: CCc1cc(Cl)c(OC)c(N2CCNCC2)c1 ; ClC[C@@H]1CO1 ; CCc1cc(Cl)c(OC)c(N2CCN(C[C@H](O)CC#N)CC2)c1]

(S)-Epichlorohydrin (46 mL, 589 mmol, 1.5 eq) was added into a mixture of 1-(3-chloro-5-ethyl-2-methoxyphenyl) piperazine 5 (100 g, 393 mmol, 1 eq) with NaHCO₃ (99 g, 1178 mmol, 3 eq) in 1000 mL EtOH at room temperature with stirring. The reaction mixture was stirred at room temperature for overnight till all starting material disappeared via LCMS. Sodium Cyanide (57.7 g, 1178 mmol, 3 eq) in 500 mL water was added one time. This mixture was heated to 40° C. for overnight. The reaction mixture was cooled to room temperature and diluted to total volume to 3000 mL by adding water (1500 mL). The mixture was extracted with ethyl acetate (2×1000 mL). The combined organic phase was washed with brine (3×500 mL) and dried over Na₂SO₄. The solvent was evaporated under reduce pressure to obtain brown oil 140 g (106%). After flash column 113.9 g (86%) clear oil was obtained. 1H NMR (400 MHz, Chloroform-d) δ 6.86 (d, J=2.0 Hz, 1H), 6.61 (d, J=2.1 Hz, 1H), 4.07-3.96 (m, 1H), 3.83 (s, 3H), 3.68 (s, 1H), 3.15 (td, J=19.2, 18.1, 8.5 Hz, 4H), 2.82 (dt, J=10.2, 4.7 Hz, 2H), 2.62 (dd, J=16.7, 5.7 Hz, 2H), 2.60-2.47 (m, 5H), 1.20 (t, J=7.6 Hz, 3H). ¹³C NMR (101 MHz, Chloroform-d) δ 146.56, 146.02, 141.05, 128.44, 122.66, 117.14, 116.86, 62.78, 62.55, 59.25, 50.45, 28.60, 23.42, 15.56.

### Example 11

This example sets forth reaction conditions for the fifth step of Scheme III in which compound 15 is formed from compound 5.

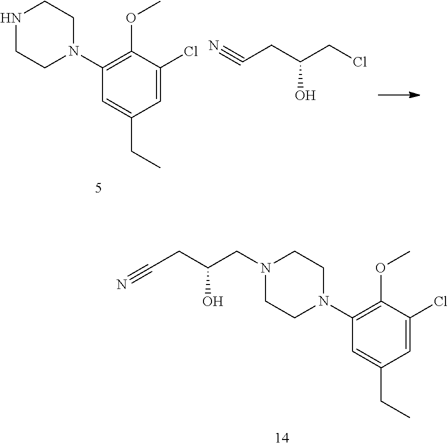

Sodium bicarbonate (33 mg, 0.393 mmol, 2 eq) was added to solution of 1-(3-chloro-5-ethyl-2-methoxyphenyl)-piperazine (50 mg, 0.196 mmol, 1 eq) and (R)-4-chloro-3-hydroxybutanenitrile (235 mg, 1.96 mmol, 10 eq) in 3 mL ethanol. This mixture was heated to 75° C. for 36 hours. The reaction was followed via LCMS. When compound 5 was total consumed, the mixture was cooled to room temperature, and 6 mL water was added. It was extracted with MTBE (3×5 mL). Combined organic phase was washed with brine (3×5 mL) and dried with Na₂SO₄. Solvent was removed under reduce pressure and crude product was purified via ISCO to obtain brown oil 53 mg (80%). ¹H NMR (400 MHz, Chloroform-d) δ 6.86 (d, J=2.0 Hz, 1H), 6.61 (d, J=2.1 Hz, 1H), 4.07-3.96 (m, 1H), 3.83 (s, 3H), 3.68 (s, 1H), 3.15 (td, J=19.2, 18.1, 8.5 Hz, 4H), 2.82 (dt, J=10.2, 4.7 Hz, 2H), 2.62 (dd, J=16.7, 5.7 Hz, 2H), 2.60-2.47 (m, 5H), 1.20 (t, J=7.6 Hz, 3H). ¹³C NMR (101 MHz, Chloroform-d) δ 146.56, 146.02, 141.05, 128.44, 122.66, 117.14, 116.86, 62.78, 62.55, 59.25, 50.45, 28.60, 23.42, 15.56.

### Example 12

This example sets forth reaction conditions for the sixth step of Schemes II and III in which compound 7 is formed from compound 14.

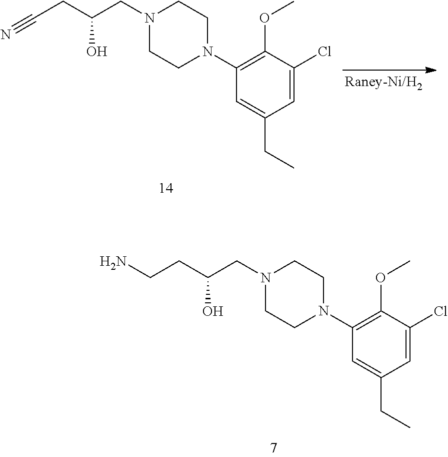 [SMILES: CCc1cc(Cl)c(OC)c(N2CCN(C[C@H](O)CC#N)CC2)c1 ; CCc1cc(Cl)c(OC)c(N2CCN(C[C@H](O)CCN)CC2)c1]

Compound 14 (8.1 g, 24 mmol) was dissolved in 100 mL 7 N NH₃ in Methanol and Ri-Ni (1 g, wet) was added and it was hydrogenated under 50 psi at room temperature for overnight. Followed via LCMS it was only 15% conversion. It was continuously hydrogenated under 50 psi at 40° C. for another 48 hours. The solvent was removed via reduced pressure. The crude oil was purified through ISCO to obtain tint oil 4.5 g (55%).

### Alternative Example 12

This example sets forth alternative reaction conditions for the sixth step of Schemes II and III in which compound 7 is formed from compound 14.

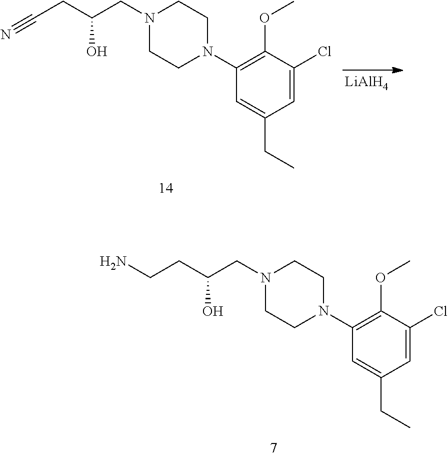

(R)-4-(4-(3-chloro-5-ethyl-2-methoxyphenyl)piperazin-1-yl)-3-hydroxybutannitrile 14 (1 g, 2.96 mmol, 1 eq) in 10 mL THF was added during15 minutes via syringe pump into a solution of LiAlH₄ (5.92 mL, 5.92 mmol, 1 M in THF, 2 eq) in 15 mL THF at −10° C. under N₂. The reaction temperature was slightly changed to −5° C. After being checked via LCMS, the reaction mixture still had around 20% compound 14 left, and another one equivalent of LAH in THF was added at this temperature. After 30 minutes it was quenched with 5N NaOH, and solid residue was washed with 2×15 mL MeOH, the filtrate was evaporated under reduce pressure to obtain brown oil (1.012 g), It was directly used for next reaction.

Any combination of disclosed features herein is considered part of the present disclosure. Each of the elements described herein, or two or more together, are also within the scope of the present disclosure. All references cited in this specification are herein incorporated by reference as though each reference was specifically and individually indicated to be incorporated by reference.

What is claimed is:

1. A method of preparing a compound 8, 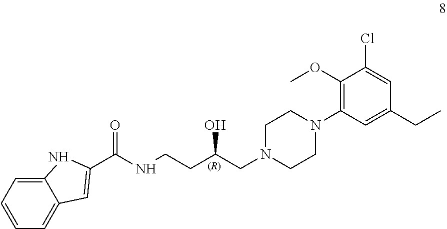
the method comprising:
coupling compound 7: 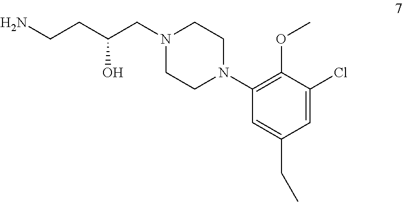 [SMILES: CCc1cc(Cl)c(OC)c(N2CCN(C[C@H](O)CCN)CC2)c1]
or a salt thereof, with 1H-indole-2-carboxylic acid in the presence of a coupling agent, wherein compound 7 is prepared by one or both of the following:
reacting compound 6 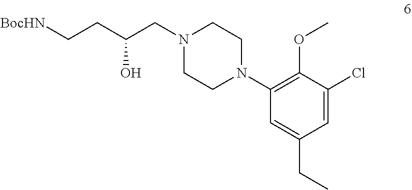 [SMILES: CCc1cc(Cl)c(OC)c(N2CCN(C[C@H](O)CCNC(=O)OC(C)(C)C)CC2)c1]
or a salt thereof, with hydrogen chloride, to provide compound 7, or a salt thereof, and
reacting a compound 14 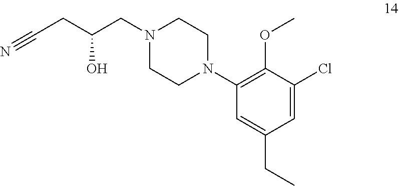 [SMILES: CCc1cc(Cl)c(OC)c(N2CCN(C[C@H](O)CC#N)CC2)c1]
or a salt thereof, with one or both of a catalyst and a reducing agent, to provide compound 7, or a salt thereof, and
wherein compound 6 is prepared by reacting compound 5: 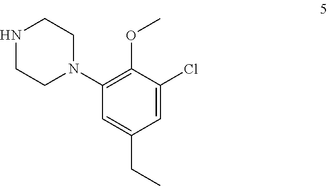 [SMILES: CCc1cc(Cl)c(OC)c(N2CCNCC2)c1]
or a salt thereof, with compound 9, Boc representing a tBuO(CO)-group:

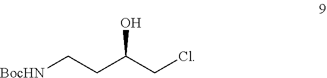 [SMILES: CC(C)(C)OC(=O)NCC[C@@H](O)CCl]

2. The method of claim 1, wherein compound 7 is prepared by reacting a compound 6 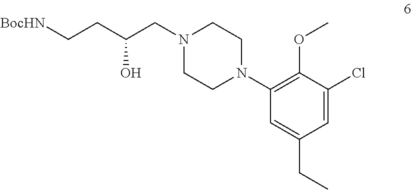 [SMILES: CCc1cc(Cl)c(OC)c(N2CCN(C[C@H](O)CCNC(=O)OC(C)(C)C)CC2)c1]
or a salt thereof, with hydrogen chloride, to provide compound 7, or a salt thereof, and
compound 6 is prepared by reacting a compound 5: 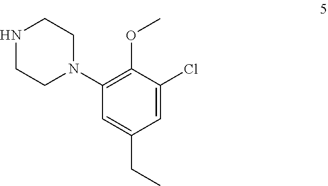 [SMILES: CCc1cc(Cl)c(OC)c(N2CCNCC2)c1]
or a salt thereof, with compound 9, Boc representing a tBuO(CO)-group:

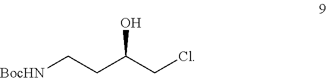 [SMILES: CC(C)(C)OC(=O)NCC[C@@H](O)CCl]

3. The method of claim 1, wherein compound 9 is prepared by reacting (R)-4-chloro-3-hydroxybutanenitrile with Boc-anhydride and one or both of a catalyst and a reducing agent, to provide the compound 9.

4. The method of claim 3, wherein the reducing agent is used for preparing compound 9.

5. The method of claim 4, wherein the reducing agent comprises lithium aluminum hydride, sodium borohydride, or hydrogen, or any combination thereof.

6. The method of claim 3, wherein the catalyst is used for preparing compound 9.

7. The method of claim 6, wherein the catalyst comprises Raney nickel, Raney cobalt, or nickel chloride, or any combination thereof.

8. The method of claim 3, wherein both the catalyst and the reducing agent are used for preparing compound 9.

9-16. (canceled)

17. The method of claim 1, wherein compound 5 is prepared by reacting a compound 4: 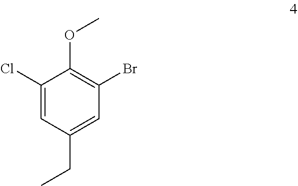
with piperazine, in the presence of a base, a diphosphine, and Pd(OAc)₂ to provide compound 5; or
reacting 2-bromo-6-chloro-4-ethylphenol with methyl iodide in the presence of base; or
both.

18. (canceled)

19. The method of claim 17, wherein 2-bromo-6-chloro-4-ethylphenol is prepared by reacting 2-chloro-4-ethylphenol with a brominating agent.

20. The method of claim 19, wherein the brominating agent is N-bromosuccinimide (NBS).

21. The method of claim 19, wherein 2-chloro-4-ethylphenol is prepared by reacting 4-ethylphenol with a chlorinating agent.

22. A compound suitable for use in the method of claim 1, the compound comprising:
the compound 9 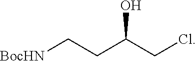 [SMILES: CC(C)(C)OC(=O)NCC[C@@H](O)CCl]
the compound 5 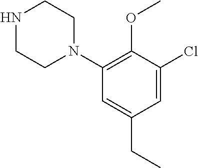 [SMILES: CCc1cc(Cl)c(OC)c(N2CCNCC2)c1]
or a salt thereof;
the compound 6 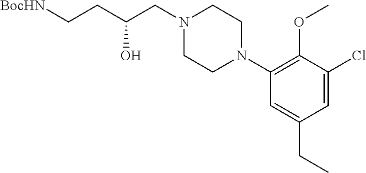 [SMILES: CCc1cc(Cl)c(OC)c(N2CCN(C[C@H](O)CCNC(=O)OC(C)(C)C)CC2)c1]
or a salt thereof;
the compound 7 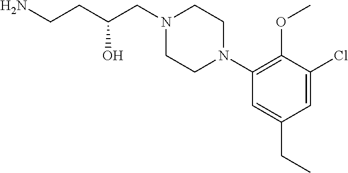 [SMILES: CCc1cc(Cl)c(OC)c(N2CCN(C[C@H](O)CCN)CC2)c1]
or a salt thereof:
the compound 7-HCl 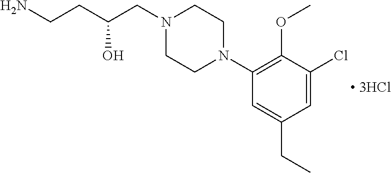 [SMILES: CCc1cc(Cl)c(OC)c(N2CCN(C[C@H](O)CCN)CC2)c1]
or a hydrate thereof;
the compound 14 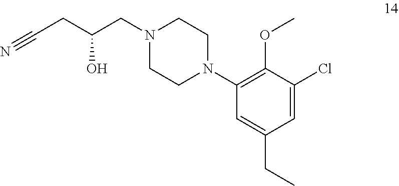 [SMILES: CCc1cc(Cl)c(OC)c(N2CCN(C[C@H](O)CC#N)CC2)c1]
or a salt thereof; or
any combination thereof.

23. A method of preparing compound 9 of claim 41, the method comprising reacting (R)-4-chloro-3-hydroxybutanenitrile with Boc-anhydride and one or both of a catalyst and a reducing agent, to provide the compound 9.

24. The method of claim 23, wherein the reducing agent is used for preparing compound 9.

25. The method of claim 24, wherein the reducing agent comprises lithium aluminum hydride, sodium borohydride, or hydrogen, or any combination thereof.

26. The method of claim 23, wherein the catalyst is used for preparing compound 9.

27. The method of claim 23, wherein the catalyst comprises Raney nickel, Raney cobalt, or nickel chloride, or any combination thereof.

28. The method of claim 23, wherein both the catalyst and the reducing agent are used for preparing compound 9.

29-40. (canceled)

41. The compound of claim 22, wherein the compound comprises compound 9.
